SEQUENCE LISTING (4 sequences)

SEQ ID NO: 1 - DNA, 122 bp, Homo sapiens
aatccaatgctgtcactccattccagacagaccgaaatacttggatggccctgatgacaccgtgttctcctgacttccttgctttcatttcatgtctcagtttaaactttgatttgctagca

SEQ ID NO: 2 - DNA, 30 bp, Homo sapiens
tgtggatagtgataatttgttataaagtaa

SEQ ID NO: 3 - DNA, 28 bp, Homo sapiens
aacctcatccaatcttaaaacacttata

SEQ ID NO: 4 - DNA, 25 bp, Homo sapiens
aatgttgttattttattttagatag

(19) United States
(12) Patent Application Publication
(10) Pub. No.: US 2020/0208203 A1
(43) Pub. Date: Jul. 2, 2020

(54) KIT FOR DIAGNOSIS OF SYSTEMIC LUPUS ERYTHEMATOSUS AND PROBE FOR THE DETECTION AND QUANTIFICATION OF THE METHYLATION LEVEL OF AN IFI44L FRAGMENT

(71) Applicants: THE SECOND XIANGYA HOSPITAL OF CENTRAL SOUTH UNIVERSITY, CHANGSHA CITY (CN); THE REGENTS OF THE UNIVERSITY OF MICHIGAN, ANN ARBOR, MI (US)

(72) Inventors: QIANJIN LU, CHANGSHA CITY (CN); MING ZHAO, CHANGSHA CITY (CN); AMR H. SAWALHA, ANN ARBOR, MI (US)

(21) Appl. No.: 16/817,729

(22) Filed: Mar. 13, 2020

Related U.S. Application Data
(62) Division of application No. 15/578,963, filed on Dec. 1, 2017.

(30) Foreign Application Priority Data
Jun. 3, 2015 (CN) 201510297277.X

(51) Int. Cl. C12Q 1/6827; C12N 15/11; C12Q 1/68; C12Q 1/6806; C12Q 1/6816; C12Q 1/6869; C12Q 1/6883

(52) U.S. Cl. CPC C12Q 1/6827; C12N 15/11; C12Q 1/68; C12Q 1/6806; C12Q 2600/156; C12Q 1/6869; C12Q 1/6883; C12Q 2600/118; C12Q 2600/154; C12Q 1/6816

(57) ABSTRACT

A kit for early diagnosis of systemic lupus erythematosus is provided. The kit for early diagnosis of systemic lupus erythematosus includes a set of primer pairs suitable for the detection and quantification of the methylation level of an IFI44L fragment, and a DNA sequence of the IFI44L fragment includes SEQ ID NO: 1.

### CROSS-REFERENCE TO RELATED PATENT APPLICATION

This application is a divisional application of U.S. application Ser. No. 15/578,963 filed on Dec. 1, 2017, and entitled “BIOMARKER AND DIAGNOSTIC KIT OF SYSTEMIC LUPUS ERYTHEMATOSUS”, now issued, the entire disclosures of which are incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present invention relates to a DNA methylation biomarker in peripheral blood from systemic lupus erythematosus, and a diagnostic kit for systemic lupus erythematosus.

### BACKGROUND OF THE DISCLOSURE

SLE (systemic lupus erythematosus, SLE) is a multi-organ, multi-system autoimmune disease characterized by variable clinical manifestations, affecting kidney, neuropsychiatric and blood systems, etc. The early diagnosis of the SLE patients is of great importance in the prevention and treatment of SLE if it can be developed before suffering pivotal organs, thereby improving the quality of life and increasing the survival rate. However, the current biological diagnostic markers are mostly detected after the organ damages occurring in the biochemical and immunological level, therefore, early diagnosis cannot be applied in the organ-involved patients of SLE.

### Technical Problem

In recent years, the apparent genetic marker research has developed rapidly, many epigenetic markers such as DNA methylation markers, serum microRNA markers were screened and identified these markers for early diagnosis and prognosis of the disease great value. A lot of literatures have identified that hypomethylation of DNA, is involved in the aberrant activation of CD4⁺T cell, thereby, plays a pivotal role in the pathogenesis of SLE. Previous studies identified hypomethylation genes in CD4⁺T cells from SLE patients include CD11a, CD70, CD40L and perforin, etc. Hypomethylation of these genes contributes to their overexpression, thus activating the autoreactive T cells, consequently, leads to the perturbance of SLE. Recently, the phenomenon of methylation in regulatory sequences of CD11a and CD70 promoter has been identified to be used as a secondary diagnosis in SLE. However, this method is limited, for detecting the methylation levels of the two genes can only be done in peripheral blood CD4⁺T cells genome. This requires us to collect more samples of peripheral blood (about 20 ml or more), and then using the density gradient centrifugation and MACS methods to isolate the peripheral blood CD4⁺T cells. In addition to large samples and low compliance of the patients, high cost and the time-consuming of the experiments, contribute most to the burden on patients. Additionally, previous methods used to detect the methylation levels of CD11a and CD70 genes, including cloning and sequencing, chip technology, all of which are time-consuming and, without a precise quantitative methylation level, bringing difficulties when applied in clinic.

To date, there is lack of diagnostic criteria for SLE with high sensitivity and specificity, autoantibodies like anti-nuclear antibodies (ANA), exhibiting high sensitivity (95%) but relatively low specificity (65%) in SLE. Due to this, the joint determination of various laboratories indexes was a supplementary diagnostic method in SLE, and resulted in the increasing medical costs and placing a heavy burden on patients. Therefore, developing a new diagnostic marker for SLE will be of great importance and necessary to improve the diagnosis and treatment level of this disease.

### SUMMARY OF THE DISCLOSURE

### Solution to Problem

### Technical Solution

The object of the present invention is to provide a new DNA methylation biomarker with high sensitive in peripheral blood from systemic lupus erythematosus patients, and accordingly to provide a diagnostic kit with high sensitivity and specificity for systemic lupus erythematosus, because traditional systemic lupus erythematosus laboratory indexes sensitivity or specificity are not high so as to overcome the deficiencies of the prior art through the present invention.

Long term studies by the inventors have found that the genome-wide epigenetic modifications may play a central role in the development of SLE, especially some aberrant DNA methylations, may be used as early diagnostic markers. It has been confirmed that the methylation levels at two CG sites in the region of IFI44L gene promoter in SLE patients were significantly reduced compared with the healthy controls and the RA disease controls in the SLE patients, when detecting DNA methylation status within 1500 bp upstream region of the IFI44L transcriptional start site through large samples of SLE patients, healthy people, and patients with RA. Hence, the methylation levels of two CG sites used for the diagnosis of sensitivity and specificity for SEL are high.

The DNA methylation marker with high sensitivity and specificity in peripheral blood from systemic lupus erythematosus patients is a DNA sequence within 1500 bp upstream from a transcription start site of a human IFI44L gene, namely chr1: 79,085,190-79,085,311 (hg19), and a DNA sequence thereof is represented by SEQ ID NO: 1.

The DNA sequence contains two CG sites, and the methylation levels thereof in peripheral blood from SLE patients were significantly reduced compared with the healthy controls and also were significantly reduced compared with the RA disease controls.

The present invention also provides use of a biomarker as defined in the DNA sequence represented by SEQ ID NO: 1 for the manufacture of SLE diagnostic kit, the use comprising detecting the methylation levels of two CG sites contained in the DNA sequence represented by SEQ ID NO: 1 in peripheral blood from subjects.

Specifically, the sequencing results analyzed by the software to determine the methylation levels of the DNA sequence within 1500 bp upstream from a transcription start site of a subject IFI44L gene in peripheral blood after sequencing by the PCR amplification of target DNA fragment, comprising the following steps: (1) genome-wide DNA extraction in peripheral blood from the subjects; (2) measuring the concentration of the extracted genomic DNA; (3) treating the genomic DNA with bisulfite; (4) amplifying the DNA fragments by the specific PCR primers; (5) examining PCR products by electrophoresis; (6) sequencing the PCR products; (7) analyzing the results from sequencing and obtaining the methylation levels of two CG sites contained in SEQ ID NO: 1.

Another object of the present invention is to provide a diagnostic kit for the diagnosis of SLE, comprising a set of PCR primers as set forth in SEQ ID NO: 2 and SEQ ID NO: 3, and a probe as set forth in SEQ ID No: 4, as well as any desired reagents or media, such as genomic DNA extraction from peripheral blood, measuring DNA concentration, bisulfite treatment, PCR analysis, electrophoresis, and pyrosequencing analysis. More specifically, one or more selected components can be involved in the diagnostic kit as follows: deoxyribonucleoside triphosphates, buffers, stabilizers, thermostable DNA polymerase and markers (including fluorescent labels, chemiluminescent labels and radioactive labels).

The methylation levels of SEQ ID NO: 1 sequence at the two CG sites detected by the present invention need to design specific PCR primers amplification of the DNA sequence of SEQ ID NO: 1 at the two CG sites. Primer design is based on the target DNA sequence including SEQ ID NO: 1 and the DNA sequence including the bases in 200 bp nucleotides upstream and downstream sequence, the primer sequences are: upstream primer 5′-TGTGGATAGTGATAATTTGTTATAAAGTAA-3′ (as shown in SEQ ID NO: 2); downstream primer 5′-AACCTCATCCAATCTTAAAACACTTATA-3′ (as shown in SEQ ID NO: 3), the downstream primer 5′-end is labeled with biotin. The methylation levels of the DNA segment at the two CG sites for pyrosequencing analysis of the present invention needs a special probe, and the primer design is based on a segment of SEQ ID NO: 1 in 1500 bp upstream region of the IFI44L transcriptional start site as primer sequences: 5′-AATGTTGTTATTTTATTTTAGATAG-3′ ((as shown in SEQ ID NO: 4).

### Advantageous Effects of Invention

### Advantageous Effects

DNA methylation chip (Illumina 450K) was firstly used to screen the differential DNA methylation of genes in peripheral blood cells from SLE patients in the worldwide. The present invention provides SLE early diagnostic kits through large-scale screening of clinical samples using the latest genetic and epigenetic detection technology to find markers for early diagnosis in patients with SLE. In comparison with the samples from healthy group, we found some hypermethylation or hypomethylation of specific genes in the peripheral blood cells from patients with SLE, among which, IFI44L gene showed significant changes in DNA methylation level. By expanding the sample of subjects, further studies have confirmed that the DNA methylation level of the IFI44L promoter in blood from SLE patients was significantly reduced compared with the normal people and RA patients. IFI44L is an IFN-inducible gene located in the type I IFN signaling pathway. IF144L can be used as a diagnostic maker for SLE as the type I IFN signaling pathway plays an important role in the pathogenesis of SLE.

The present invention overcomes the above noted deficiencies and can be carried out by no more than 1 ml peripheral blood from SLE patients. The patient's compliance can be largely improved by DNA methylation markers. The diagnostic kit of the present invention detects with high specificity and sensitivity and has followed advantages: less time consuming, simple operation and small amount of sample required for easy on widespread clinical application prospect. The accuracy and specificity of the detecting results (which are over 90%), as well as the efficiency have been greatly improved when using pyrosequencing instrument with specific primers and probes. The development and application of the invention will be of great importance for improving the diagnosis and treatment of patients with SLE, ultimately improving the quality of life and increasing the survival rates.

### BRIEF DESCRIPTION OF THE DRAWINGS

### Brief Description

The present disclosure will become more fully understood from the following detailed description and accompanying drawings.

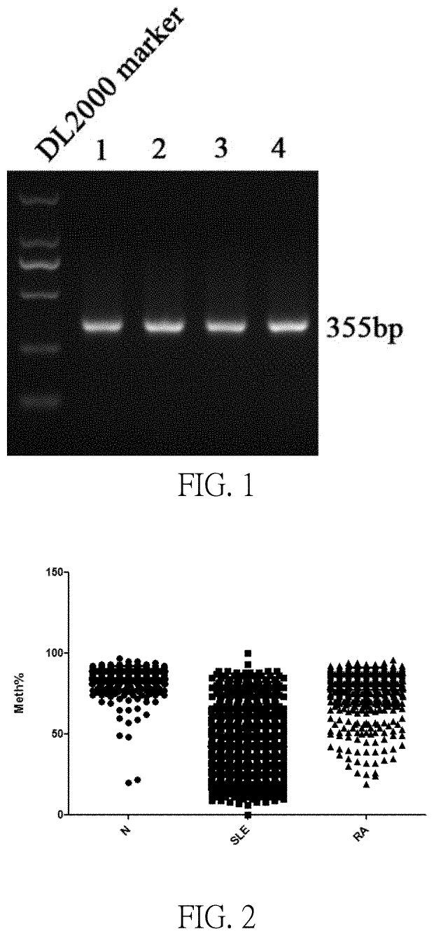

FIG. 1 is the specific primer PCR amplified DNA fragment (SEQ ID NO: 1) of electrophoresis.

FIG. 2 is the differences of methylation level at CG site 1 in SLE patients, healthy controls and RA patients.

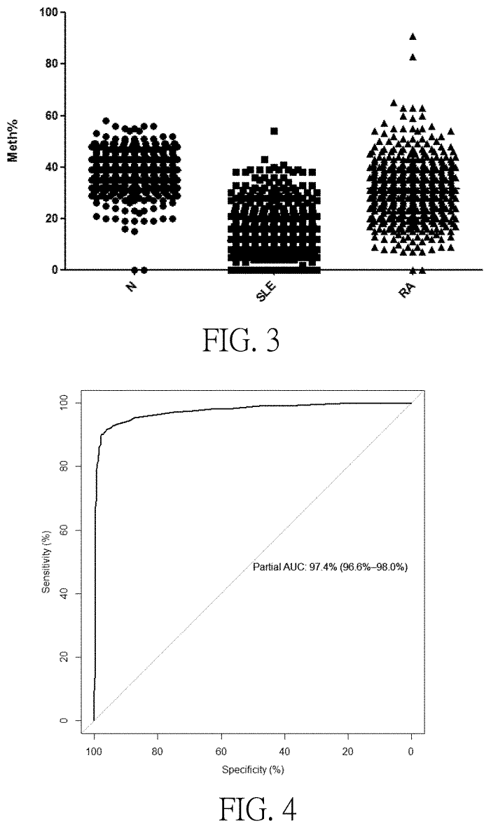

FIG. 3 is the differences of methylation level at CG site 2 in SLE patients, healthy controls and RA patients.

FIG. 4 is the ROC graph of methylation at level CG site 1 for SLE diagnosis (compared with healthy controls).

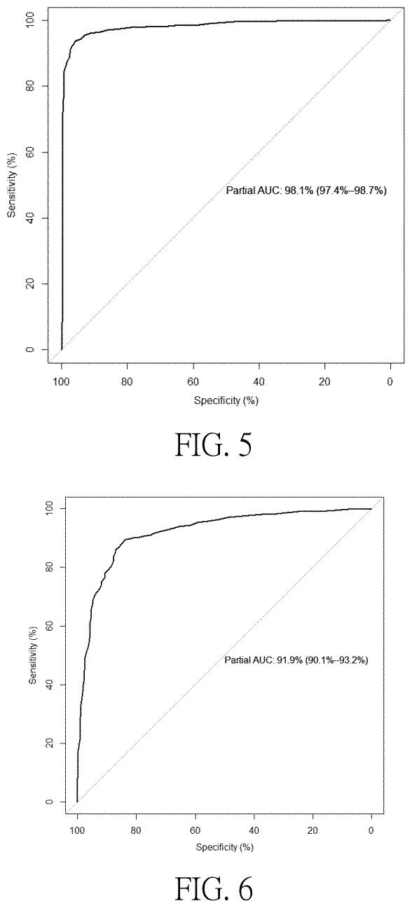

FIG. 5 is the ROC graph of methylation at level CG site 2 for SLE diagnosis (compared with healthy controls).

FIG. 6 is the ROC graph of methylation level at CG site 1 for the differential diagnosis between SLE and RA (compared to RA).

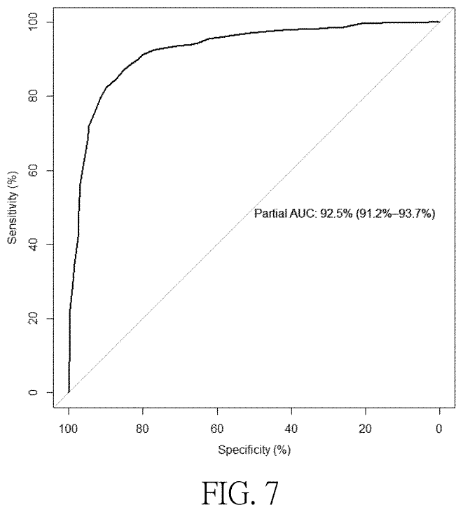

FIG. 7 is the ROC graph of methylation level at CG site 2 for the differential diagnosis between SLE and RA (compared with RA).

### DETAILED DESCRIPTION OF THE EXEMPLARY EMBODIMENTS

### Example

### Embodiments of the Present Invention

The following is the detail descriptions of the embodiments of the present invention, including but not limited to, those technological changes according to the invention are protected. Unless otherwise stated, all the technical and scientific terms used herein are generally known to general technical staff in this field.

### Example 1: Preparation of SLE Diagnostic Kit

The present invention provides a diagnostic kit for SLE consists of: (1) Whole Blood DNA extraction reagents: proteinase K, cell lysate, wash buffer, elution buffer, adsorption column; (2) bisulfite treatment reagents: dilution buffer, conversion buffer, binding buffer, wash buffer, de-sulfonation buffer, elution buffer; (3) PCR Reagents: DNA polymerase, PCR reaction buffer, PCR primers in SEQ ID NO: 2 and SEQ ID NO: 3; (4) electrophoresis reagents of PCR products: the electrophoresis buffer and agarose; (5) pyrosequencing reagents: streptomycin labeled agarose beads, denaturing buffer, sequence primers shown in SEQ ID NO: 4, washing buffer; (6) software for sequencing analysis: PyroMark Q24 Application Software 2.0.

### Example 2: The Application of the Diagnostic Kit for SLE Patients and Detection of DNA Methylation Levels in Peripheral Blood

Step 1: SLE Patient Peripheral Blood Genomic DNA Extraction

(1) Add 0.5 ml whole blood to a 1.5 ml micro-centrifuge tube, and then add 1 ml of ice cold nuclease free water, mix thoroughly by vortexing or pipetting; (2) Incubate the sample for 5 min at room temperature and Centrifuge for 5 min at 800×g (˜3,000 rpm); (3) Discard the supernatant and resuspend the pellet in 150 μl of 1×PBS; (4) Add 20 μl of Proteinase K Solution, mix by vortexing; (5) Add 350 μl of Lysis Solution, mix thoroughly by vortexing or pipetting; (6) Incubate the sample at 56° C. for 10 minutes during which the sample and mix by inverting 3 times; (7) Add 180 μl of ethanol (96-100%) and mix by pipetting; (8) Transfer the prepared mixture to the spin column. Centrifuge for 1 min at 6,000×g (˜8,000 rpm); (9) Place the column into a new collection tube; (10) Add 500 μl of Wash Buffer WB I. Centrifuge for 1 min at 8,000×g (˜10,000 rpm). Discard the flow-through and place the column back into the collection tube; (11) Add 500 μl of Wash Buffer II to the column. Centrifuge for 3 min at maximum speed (≥20,000 g, ≥14,000 rpm). Discard the collection tube containing the flow-through solution; (12) ≥20000 g (≥14000 rpm) empty centrifugal 1 min, the adsorption column was transferred to a new 1.5 ml centrifuge tube; (13) Add 80 μl of Elution Buffer to the center of the column membrane to elute genomic DNA. Incubate for 2 min at room temperature; (14) Centrifuge for 1 min at 8,000×g (˜10,000 rpm); (15) Collect genomic DNA.

Step 2: Determination of the Concentration of Extracted Genomic DNA.

Using the NanoDrop 2000 software from the Thermo Scientific, drawing 1 μl DNA extracts to the detecting board and reading the concentration of the sample.

Step 3: Bisulfite Treatment of Genomic DNA.

(1) According to the DNA concentration and calculate the volume of bisulfite treated DNA (200 g); (2) Add 5 μl of M-Dilution Buffer to the DNA sample and adjust the total volume to 50 μl with water. Mix the sample by flicking or pipetting up and down; (3) Incubate the sample at 37° C. for 15 minutes; (4) Prepare the CT Conversion Reagent (CT), minimize its exposure to light: Add 750 μl water and 210 μl of M-Dilution Buffer to a tube of CT Conversion Reagent, mix at room temperature with frequent vortexing or shaking for 10 minutes; (5) After the above incubation, add 100 μl of the prepared CT Conversion Reagent to each sample and mix; (6) Incubate the sample in the dark at 50° C. for 12-16 hours; (7) Incubate the sample at 0-4° C. (e.g., on ice) for 10 minutes; (8) Add 400 μl of M-Binding Buffer to a column and place the column into a provided Collection Tube; (9) Load the sample (from Step 7) into the column containing the M-Binding Buffer. Close the cap and mix by inverting the column several times; (10) Centrifuge at full speed (>10,000×g) for 30 seconds; (11) Add 100 μl of M-Wash Buffer to the column. Centrifuge at full speed for 30 seconds. Discard the supernatant; (12) Add 200 μl of M-Desulphonation Buffer to the column and let stand at 20-30° C. for 18 minutes; (13) Centrifuge at full speed (>10,000×g) for 30 seconds; (14) Add 200 μl of M-Wash Buffer to the Wash Buffer. Centrifuge at full speed (>10,000 xg) for 30 seconds; (15) Repeat the previous steps once. Discard the supernatant; (16) Centrifuge at full speed (>10,000×g) for 30 seconds. Discard the supernatant; (17) Place the column into a 1.5 ml micro-centrifuge tube; (18) Add 10 μl of M-Elution Buffer directly to the column matrix. Centrifuge for 30 seconds at full speed (>10,000×g) to elute the DNA; (19) Repeat the previous step once, discard the column. The column collected after the centrifuge tube is the DNA treated with sulfite.

Step 4: Amplifying Target DNA Fragment and Sequencing

These include:

1. Design specific PCR primers to amplify the target DNA fragment followed by sequencing.

Use PyroMark Assay Design 2.0 software to design primers. Input the target DNA sequence (including the bases in 200 bp upstream and downstream sequence) into software, and then obtaining specific PCR primers, upstream primer 5′-TGTGGATAGTGATAATTTGTTATAAAGTAA-3′ (as shown in SEQ ID NO: 2), downstream primer 5′-AACCTCATCCAATCTTAAAACACTTATA-3′ (as shown in SEQ ID NO: 3), which can amplify a PCR product containing the target DNA sequence, with biotin markers at 5 ′end of the downstream primer. The sequencing results are average at a length of 355 bp, while two CG sites can be detected in the target DNA sequence. The probe sequence is designed as: 5′-AATGTTGTTATTTTATTTTAGATAG-3′ (as shown in SEQ ID NO: 4).

DNA fragments by specific PCR primers amplification.

The PCR components are shown in table 1; and the PCR reaction conditions are shown in table 2.

TABLE 1
PCR components

| | | |
|---|---|---|
| 5 × buffer | 4 | μl |
| Upstream primer (10 ng/μl) | 0.4 | μl |
| Downstream primer (10 ng/μl) | 0.4 | μl |
| dNTP | 0.5 | μl |
| Taq DNA polymerase | 0.5 | μl |
| Nuclease-Free Water | 12.2 | μl |
| DNA | 2 | μl |
| Total volume | 20 | μl |

TABLE 2
Specific PCR conditions of Methylation

| | | | | Cycle |
|---|---|---|---|---|
| Step | Temperature | Time | | number |
| Initial | 96° C. | 2 | min | 1 |
| activation | | | | |
| Denaturation | 96° C. | 10 | s | 45 |
| Annealing | 58° C. | 30 | s | |
| Extension | 72° C. | 1 | min | |
| Final | 72° C. | 10 | min | 1 |
| extension | | | | |

3. Detect PCR products by agarose gel electrophoresis.

(1) prepare electrophoresis buffer (50×TAE) and dilute to 1×TAE with deionized water; Weigh out the 0.5 g agarose and place in an Erlenmeyer flask with 50 ml 1×TAE. Dissolve the agarose in a revolving-plate microwave oven. (2) Cool the solution to 50° C., add 2.5 μl Ethidium bromide (EB), swirl and mix, then pour the gel onto a taped plate with casting combs in place immediately. Allow more than 60 minutes for solidification. (3) Carefully remove the tape and the gel casting combs, place the gel in a horizontal electrophoresis apparatus. Add 1×TAE electrophoresis buffer to the reservoirs until the buffer just covers the agarose gel. (4) Add 6 μl PCR products into each wells and load DNA marks into the first well. (5) Electrophorese the gel at 150-200 mA/135V until the required separation has been achieved, usually 25 minutes. (6) After electrophoresis, the gel was placed condensate gel imaging system to observe any obvious specific electrophoretic bands, as shown in FIG. 1.

Apply Qiagen pyrosequencing Q24 to DNA sequence.

(1) Prepare the reagents and samples; 50 ml ethanol (70%) (15 ml+35 ml), 40 ml Denaturation Solution, 10 μm sequencing primers, sequencing DNA probe sequence 5′-AATGTTGTTATTTTATT TTAGATAG-3′, as shown in SEQ ID NO: 4. 50 ml wash buffer (45 ml H2O+5 ml Wash Buffer), wash reagent compartment (each with up to thirty times), five high-water. (2) Prepare a mixture called Mix\textcircled{1}: total volume is 80 μl/well, including 40 μl binding buffer+2 μl streptavidin-coated Sepharose beads+18 μl of high purity water+20 μl PCR product (final plus). (3) Prepare a mixture called Mix\textcircled{2}: 10 μM×X μl=0.3 μM×2 5 μl×n (n is the number of samples required, a Mix\textcircled{2} is required every 10 samples added), calculate X (requiring 10 μM sequencing primers), and 25 μl×n−X μl is the volume of annealing buffer. (4) Biotinylated PCR products are immobilized on streptavidin coated Sepharose beads. Gently shake the bottle with streptavidin-coated Sepharose beads from side to side until a homogenous solution is obtained. (5) Mix the total amount of streptavidin-coated Sepharose beads (2 μl per sample) and Binding Buffer (40 μl per sample) in a tube. (6) Add high-purity water to a total volume of 80 μl per well including the PCR product to be added in step 4. The amount of water depends on the amount of PCR product used. (7) Add the solution prepared in step 2 to a 24-well PCR plate or strips. (8) Add 5-20 μl of a well-optimized, biotinylated PCR product to each well of the PCR plate (or strips) according to the plate setup. The total volume per well should be 80 μl. (9) Seal the PCR plate to ensure that no leakage is possible between the wells. (10) Agitate the PCR plate (or strips) constantly for at least 5-10 min using a mixer (1400 rpm). Sepharose beads sediment quickly and capturing of beads must take place immediately once the agitation is complete. During immobilization, prepare the vacuum workstation for the sample preparation. (11) Dilute the sequencing primer to 0.3 μM in Annealing Buffer, i.e., Mix\textcircled{2}. Add 25 μl of the solution to each well of a PyroMark Q24 Plate that is to be used. Use one of the supplied PyroMark Q24 Plate Holders as support when preparing and moving the plate.

Procedures of Vacuum Workstation: (1) Ensure that the vacuum Q24 workstation correctly and securely fitted, the base plate 24 preheated (80° C.), washing trough, filling the trough (50 ml 70% ethanol, 40 ml denaturing solution, wash buffer 50 ml, 50 ml and 70 ml high purity water), to open the vacuum pump, the vacuum switch is open, a vacuum is applied in the vacuum apparatus. (2) High water probe, the vacuum is applied, the cleaning process of the probe was filtered off, washed with 70 ml high purity water probe, to ensure that the water is transferred to a waste container, the vacuum Jian shut off the vacuum, and placed the rest position (P position). (6) with 70 ml high purity water to refill the reagent tank 5. (3) 11.4.8 After the fixed sample and 11.5 ready Pyromark Q24 orifice Mix\textcircled{2}, place the PCR plate (or the strips) and PyroMark Q24 Plate on the worktable to ensure consistency with the loading position of the sample plate inch. (4) Switch on the vacuum pump, vacuum applied in the vacuum apparatus. Carefully lower the filter probes into the PCR plate (or strips) to capture the beads containing immobilized template. Hold the filter probes in place for 15 s, carefully remove the vacuum device (agar microspheres precipitate quickly, if the oscillating plate or tube is placed over row 1 mm, then again one minute before capture oscillation) to ensure that all liquid tank hole and suck out all the microspheres have been captured by filtration probe tip, 70% ethanol reagent tank 5 second. (5) The elution buffer 10 s. Raise the vacuum means exceed 90° C. vertical 5 second, to filter probe drainage, vacuum means to hold Pyromark Q24 plate, turn off the vacuum switch (OFF) on the unit by gently rocking vacuum device to release the beads i.e. probes containing the sequencing primer well plates, the vacuum switch closed (OFF), transferred to a vacuum device comprising a reagent of high purity water bath and shaken 10 second, the probe is lowered to the second reagent vessel containing pure water and a high vacuum is applied cleaning the probe, with the probe was filtered 70 ml high purity water rinsing, vacuum 90° C. raising vertical 5 second, drain probe to filter, and then close the vacuum apparatus, placing the rest position, such as a more than one orifice, again filling agent tank, repeating. Turn off the vacuum pump, and the PyroMark Q24 Vacuum Workstation has been assembled correctly and securely.

Using the PyroMark Q24. The concrete step is: (1) Annealing of sequencing primer to samples: Heat the PyroMark Q24 Plate containing the samples at 80° C. for 2 min using the PyroMark Q24 Plate Holder (two are supplied with the vacuum workstation) and a heating block. Remove the plate from the plate holder and allow the samples cool to room temperature (15-25° C.) for at least 5 min. The plate can now be processed in the PyroMark Q24 Instrument. (2) Preparation of PyroMark Gold Q24 Reagents. Open the PyroMark Gold Q24 Reagents box and remove the vials containing freeze-dried enzyme and substrate mixtures, and the tubes containing nucleotides. Reconstitute the volumes of reagents required according to the handbook supplied with the reagents and fill PyroMark Q24 Cartridge. (3) Starting the run of PyroMark Q24: Open the cartridge gate and insert the filled reagent cartridge, iInsert the USB stick containing the run file into the USB port at the front of the instrument, using the up and down screen buttons, select “Run” in the main menu and press “OK”, select the run file using the up and down screen buttons. To view the contents of a folder, select the folder and press “Select”. To go back to the previous view, press “Back”. When the run file is selected, press “Select” to start the run. When the run is finished and the instrument confirms that the run file has been saved to the USB memory stick, press “Close”. Clean the reagent cartridge.

Step 5: Analysis of methylated DNA sequence level by sequencing results.

According to FIG. sequencing results, direct reading software PyroMark value Q24 Application Software 2.0 is given to determine the methylation levels of two CG sites.

### Example 3: to Test Sensitivity and Specificity of the Diagnostic Kit for SLE

Using the method described in Example 2, detected 1056 cases of SLE patients with 587 healthy controls, 553 cases of rheumatoid arthritis (abbreviated RA) DNA sequences in patients with IFI44L gene transcription start site upstream within −1500 bp that methylation level SEQ ID NO: 1 contains two CG sites, test results show: the healthy control group, two CG sites showed hypermethylation, two CG sites methylation levels in SLE patients compared with healthy controls and RA patients were significantly lower (as shown in FIGS. 2 and 3).

To evaluate the sensitivity and specificity of methylation levels valued by ROC curves in the diagnosis of SLE. The actual area of the Area Under Curve (AUC) is from 0.5 to 1, and it is generally believed that for a diagnostic test, when the area is between 0.5 and 0.7, it is of a low diagnostic value, while the area is between 0.7 to 0.9, the diagnostic value is moderate, or of a high diagnostic value when the area is over 0.9. The analysis of methylation levels at the CG site 1 between SLE patients and healthy controls, with a specificity of 96.10895% and a sensitivity of 91.67513% (as shown in FIG. 4). The analysis of methylation levels at the CG site 2 between SLE patients and healthy controls, with a specificity of 95.91440% and a sensitivity of 93.50254% (as shown in FIG. 5). The analysis of methylation levels at the CG site 1 between SLE patients and the RA disease controls, with a specificity of 83.72549% and a sensitivity of 91.67513% (as shown in FIG. 6). The analysis of methylation levels at the CG site 2 between SLE patients and the RA disease controls, with a specificity of 89.80392% and a sensitivity of 82.53807% (as shown in FIG. 7).

### INDUSTRIAL APPLICABILITY

The present invention provides a diagnostic kit SLE overcomes the deficiencies of the prior art methods of detecting SLE, only need to extract in patients with no more than 1 ml to find DNA methylation markers from peripheral blood of SLE patients, thus significantly improve patient treatment compliance. High specificity and sensitivity of the kit of the present invention, the inspection took short, simple operation, small amount of sample required for easy on widespread clinical application prospect. Use pyrosequencing instrument with specific primers and probes can greatly reduce DNA methylation inspection time, greatly improve the efficiency and results of laboratory tests to check the accuracy and specificity (can reach more than 90%). The new technology and product development and application will have a great significance for improving the diagnosis and treatment of SLE, the SLE patients to improve quality of life and survival.

What is claimed is:

1. A kit for early diagnosis of systemic lupus erythematosus, comprising:
a set of primer pairs suitable for the detection and quantification of the methylation level of an IFI44L fragment, wherein a DNA sequence of the IFI44L fragment includes SEQ ID NO: 1.

2. The kit for early diagnosis of systemic lupus erythematosus according to claim 1, wherein the IFI44L fragment includes at least two GC sites.

3. The kit for early diagnosis of systemic lupus erythematosus according to claim 1, wherein the set of primer pairs comprise a first primer and a second primer, wherein a DNA sequence of the first primer includes SEQ ID NO: 2, and a DNA sequence of the second primer includes SEQ ID NO: 3.

4. The kit for early diagnosis of systemic lupus erythematosus according to claim 3, wherein a 5′-end of the second primer is labeled with a biotin.

5. The kit for early diagnosis of systemic lupus erythematosus according to claim 1, further comprising a probe, wherein a DNA sequence of the probe includes SEQ ID NO: 4.

6. The kit for early diagnosis of systemic lupus erythematosus according to claim 1, further comprising one or more of buffers, wash reagents, polymerases, internal controls, or reagents capable of detecting the presence of a bound nucleic acid primer.

7. A probe for the detection and quantification of the methylation level of an IF144L fragment, wherein a DNA sequence of the IFI44L fragment includes SEQ ID NO: 1, and a DNA sequence of the probe includes SEQ ID NO: 4.
